(19) United States
(12) Patent Application Publication
(10) Pub. No.: US 2004/0175471 A1
(43) Pub. Date: Sep. 9, 2004

(54) Multiple injection solutions

(75) Inventors: David W. Alexander, Boone, IA (US); Douglas N. McCloskey, Runnells, IA (US)

Correspondence Address: ZARLEY LAW FIRM P.L.C., CAPITAL SQUARE, 400 LOCUST, SUITE 200, DES MOINES, IA, 50309-2350, US

(73) Assignee: Townsend Engineering Company, Des Moines, IA (US)

(21) Appl. No.: 10/800,927

(22) Filed: Mar. 15, 2004

Related U.S. Application Data
(62) Division of application No. 10/322,838, filed on Dec. 18, 2002.

(51) Int. Cl. G01N 33/02

(52) U.S. Cl. 426/281

(57) ABSTRACT

The present invention provides an apparatus and method of injecting a brine solution into a meat product. The process includes the steps of providing high concentration brine to a brine source connected to injection heads designated for fat areas and low concentration brine to the brine source connected to injection heads designated for lean areas, and injecting the meat product with fluid in accordance with the location of lean and fat areas.

### BACKGROUND OF THE INVENTION

It is common to inject brine or similar fluids into meat products for purposes of preservation and flavoring. Many machines have been devised which have a battery of needles which are adapted to inject fluid into the meat products through the needles.

The absorption of brine in a meat product is complicated by variations in the lean content of the meat. Low lean (or fat) areas tend not to absorb as much fluid as high lean areas. For example, a 90% lean area on meat product will absorb approximately 10% of any given solution while a 50% lean area will absorb approximately 5% of any given solution. However, existing machines lack the ability to effectively inject brine into the meat product to result in a uniform distribution of brine ingredients through the meat product, particularly when variations in the lean content of the meat products exist.

Therefore, a principal object of this invention is to provide a method and means for injecting fluids into meat products which will result in a uniform distribution of brine ingredients through the meat product.

These and other objects will be apparent to those skilled in the art.

### SUMMARY OF THE INVENTION

The present invention provides an apparatus and method of injecting a brine solution into a meat product. The process includes the steps of providing high concentration brine to a brine source connected to injection heads designated for fat areas and low concentration brine to the brine source connected to injection heads designated for lean areas, and injecting the meat product with fluid in accordance with the location of lean and fat areas.

### DESCRIPTION OF THE DRAWINGS

FIG. 1 is a plan view of the device of this invention; and

FIG. 2 is a side elevation view of the device of this invention.

### DESCRIPTION OF THE PREFERRED EMBODIMENT

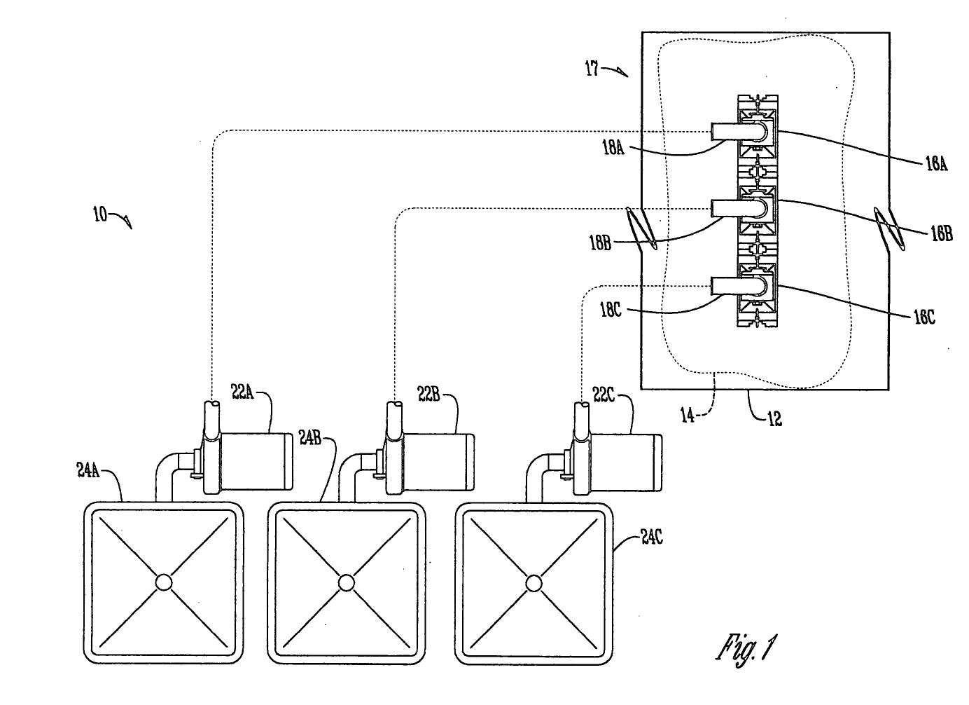

With reference to FIG. 1, a meat injection machine 10 of this invention has a horizontal conveyor 12 for intermittently longitudinally moving meat products 14 to be injected. A plurality of injection heads 16(A, B, and C) form an injection zone 17 and are operatively associated with a corresponding plurality of manifolds 18(A, B, and C) above the conveyor 12.

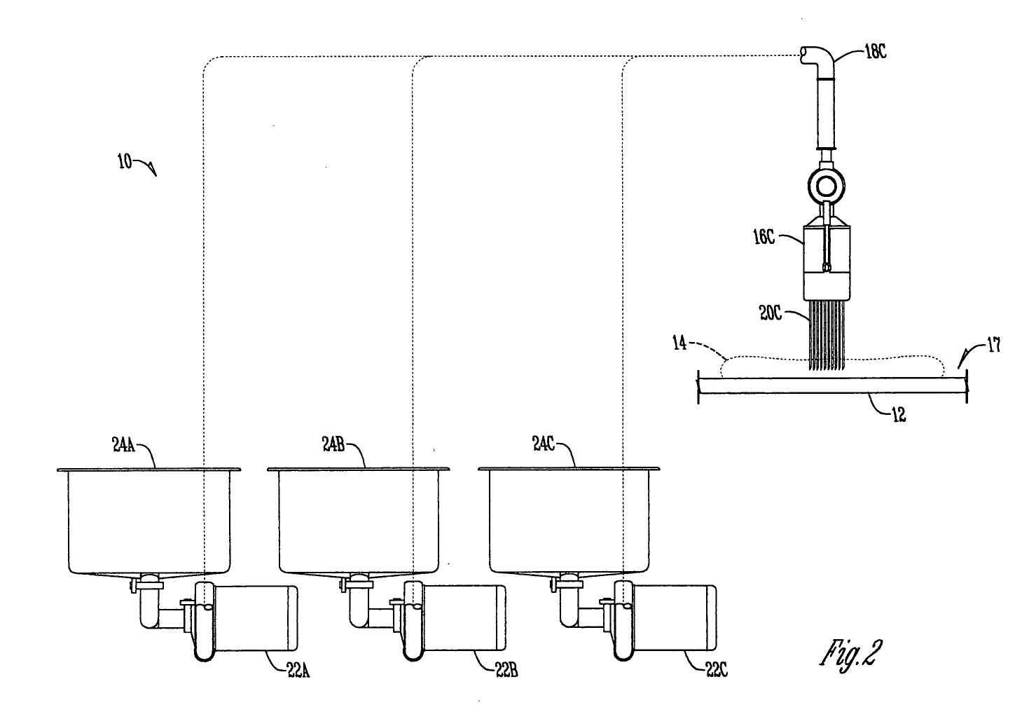

With reference to FIG. 2, the injection head 16C has associated therewith a plurality of vertically disposed injection needles forming a bank of injection needles 20C which inject fluid into meat products 14. Each injection head 16C (16A and 16B not shown) has associated therewith a bank of injection needles 20C (20A and 20B not shown).

Referring to FIG. 1 and FIG. 2, a separate fluid pump 22(A, B, and C) is connected to each of the heads 16(A, B, and C) for supplying pressurized fluids to the needles 20C (20A and 20B not shown). Each of the heads 16(A, B, and C) are connected to a separate reservoir 24(A, B, and C) of brine fluid via the pump 22(A, B, and C). The concentration of brine is different in each reservoir 24.

It will be understood that additional rows of injection heads 16 with separate reservoirs 24 can be provided for the meat injection machine 10 without departing from the present invention.

In operation, meat products 14 (pork bellies for example) are placed upon the in-feed side of the machine 10. As the meat product 14 advances on the conveyor 12 into the injection zone 17, the individual injection heads 16 come into contact with the meat product 14. As the injection heads 16 come into contact with the meat product 14, the needles 20 penetrate the meat product 14. The needles 20 subsequently inject solution into the meat product 14.

The meat product 14 is visually inspected and/or historical data is used to determine the location of lean and fat areas in the meat product 14. According to this information, the brine solution supplied to each reservoir 24 is formulated to have the optimum salt and nitrite concentration for the lean and fat distribution for the particular meat product 14 to be treated. As stated before, fat areas tend not to absorb as much fluid as lean areas. The brine solution supplied to each reservoir 24 will be formulated so that, given the same pressures from pumps 22, each head 16 will inject brine with varying concentrations of salt and nitrites where they are needed. For example, a 90% lean area on meat product 14 will absorb approximately 10% of any given solution while a 50% lean area will absorb approximately 5% of any given solution. Accordingly, the present invention would supply a brine solution to the 50% lean area at twice the concentration as the brine solution supplied for the 90% lean area. Thus, the present invention will be understood to provide a more uniform distribution of brine ingredients throughout the meat product 14.

It is therefore seen that the present invention provides a method and means for injecting fluids into meat products 14 which results in a uniform distribution of brine through the meat product 14.

It is therefore seen that this invention will accomplish at least all of its stated objectives.

I claim:

1. A method of injecting a brine solution into a meat product, comprising:
providing an injection zone comprised of a plurality of separate brine injection heads each with banks of injection needles over a meat conveyor positioned longitudinally underneath the injection heads,
connecting each of the heads and the needles therein to a separate source of brine fluid with the concentration of brine being different in each source,
providing high concentration brine to the brine source connected to heads designated for fat areas and low concentration brine to the brine source connected to heads designated for lean areas,
passing the single piece of meat product on the conveyor through the injection zone underneath the injection heads, and
causing the banks of injection needles to move downwardly to inject the meat product with fluid in accordance with the location of lean and fat areas.

2. The method of claim 1, wherein a separate fluid pump is connected to each head.

3. The method of claim 1, wherein the brine supplied from each fluid source being at the same pressure.

4. A meat injection apparatus, comprising:
a horizontal conveyor for intermittently longitudinally moving meat products to be injected,
a plurality of injection heads located over the conveyor,
a bank of vertically disposed injection needles on each head,
a plurality of fluid reservoirs in fluid flow communication with the heads and the needles therein to supply source of brine fluid to the needles, wherein the concentration of brine being different in each reservoir,
wherein the banks of injection needles move downwardly to inject the meat product by having an injection head connected to a reservoir having a high concentration of brine to penetrate an area of fat, and by having an injection head connected to a reservoir having a lower concentration of brine to penetrate an area of lean.

5. The apparatus of claim 4, wherein a separate fluid pump is connected to each head.
